(19) United States
(12) Patent Application Publication
(10) Pub. No.: US 2025/0076236 A1
(43) Pub. Date: Mar. 6, 2025

(54) METHOD FOR DETERMINING THE VOLUME FRACTION OF A PHASE OF A MULTIPHASE MEDIUM AND CORRESPONDING MEASURING DEVICE

(71) Applicant: KROHNE Messtechnik GmbH, Duisburg (DE)

(72) Inventors: Jan FOERSTER, Essen (DE); Martin KRAWCZYK-BECKER, Herne (DE); Paul WACKER, Duisburg (DE)

(73) Assignee: KROHNE Messtechnik GmbH, Duisburg (DE)

(21) Appl. No.: 18/819,340

(22) Filed: Aug. 29, 2024

(30) Foreign Application Priority Data
Aug. 29, 2023 (DE) 10 2023 123 216.9

(51) Int. Cl. G01N 27/08; G01N 33/28

(52) U.S. Cl. CPC G01N 27/08; G01N 33/2847

(57) ABSTRACT

A method for determining the volume fraction of a phase of a multiphase medium, wherein the medium has a first phase with a first value for a first electrical parameter and, at least temporarily, a second phase with a second value for the first electrical parameter. The medium is impinged with a plurality of electrical excitation signals via a pair of electrodes and a plurality of corresponding electrical response signals are captured for the electrical excitation signals. A plurality of values for the first electrical parameter of the medium is determined from the plurality of excitation signals and the plurality of response signals. The volume fraction of the second phase in the medium is determined by identifying a scattering value of a statistical scattering measure from the plurality of determined values and by using a mathematical relationship between the statistical scattering and a volume fraction of the second phase.

This nonprovisional application claims priority under 35 U.S.C. § 119(a) to German Patent Application No. 10 2023 123 216.9, which was filed in Germany on Aug. 29, 2023, and which is herein incorporated by reference.

### BACKGROUND OF THE INVENTION

### Field of the Invention

The invention relates to a method for determining the volume fraction of a phase of a multiphase medium, wherein the medium has a first phase with a first value for a first electrical parameter and, at least temporarily, a second phase with a second value for the first electrical parameter, wherein the medium is impinged with a plurality of electrical excitation signals via a pair of electrodes and a plurality of corresponding electrical response signals are captured for the electrical excitation signals, wherein a plurality of values for the first electrical parameter of the medium is determined from the plurality of excitation signals and the plurality of response signals. Furthermore, the invention also relates to a measuring device with a measurement volume serving to accommodate a medium, wherein the medium has a first phase with a first value for a first electrical parameter and, at least temporarily, a second phase with a second value for the first electrical parameter, with a control and evaluation unit, with electrodes in contact with the medium, wherein, in the operating state of the measuring device, the control and evaluation unit impinges a plurality of electrical excitation signals on the medium via the pair of electrodes and captures a plurality of corresponding electrical response signals for the electrical excitation signals, wherein a plurality of values for the first electrical parameter of the medium is determined from the plurality of excitation signals and the plurality of response signals.

### Description of the Background Art

Methods and measuring devices of the aforementioned type are often used in conjunction with other measuring methods, such as flow measurement. In flow measurement, a volumetric flow is calculated—also indirectly via the flow velocity of the medium (for example with magnetic-inductive flowmeters) or also a mass flow (for example with Coriolis mass flowmeters). If it is known that the medium can have several phases in the measuring task, then it is obvious that not only the volume flow or the mass flow of the multiphase medium is of interest, but also the composition of the medium, i.e. the proportions of the different phases in the multiphase medium. The term phase is therefore not to be understood here in a restrictive sense in the sense of an aggregate state, but rather as a multi-material system. Such multi-material systems are found in many applications in the chemical industry, in oil and gas extraction, wastewater processing, etc.

### SUMMARY OF THE INVENTION

It is therefore an object of the present invention to further develop a method and also a measuring device for determining a volume fraction of a phase of a multiphase medium in such a way that it is less sensitive to a change in the phases involved and their values for the first electrical parameter.

The object derived above is achieved in a method for determining the volume fraction of a phase of a multiphase medium and in a measuring device. The method according to the invention is characterized in that a scattering value of a statistical scattering measure is determined from the plurality of determined values for the first electrical parameter of the medium and that the volume fraction of the second phase in the medium is determined using the determined scattering value of the first electrical parameter using a mathematical relationship between the statistical scattering measure of the first electrical parameter and a volume fraction of the second phase in the medium. What is remarkable about the method is that it is not dependent on the direct values of the first electrical parameter (such as the determined electrical conductivity of the medium or the impedance of the medium) and that instead a statistical scattering measure of the first electrical parameter is used on the basis of a plurality of values for the first electrical parameter. According to the invention, it has been recognized that there is a relationship between scattering values of this statistical scattering measure and the volume fraction of the second phase in the medium. The use of such a correlation in itself already reduces the dependence on the absolute values of the first electrical parameter of the medium.

The excitation signal can be an electrical voltage or an electrical current. Accordingly, the electrical response signal can be an electrical current or an electrical voltage, in particular wherein the excitation signal can be a harmonic oscillation with an excitation frequency. The choice of the excitation signal as a harmonic oscillation enables the evaluation of first electrical parameters, which may be defined as harmonic alternating quantities.

Impingement of the medium with the electrical excitation signals can take place in a time domain in which no other measured value acquisition is performed in the medium. This measure prevents different measured value acquisitions from influencing each other and thus leading to a poorer overall measured value result. If the method is carried out with a magnetic-inductive flowmeter which—as previously carried out—already has corresponding electrodes in active connection with the medium, then the method for determining the volume fraction of a phase is performed if no magnetic-inductive flowmeter measurement is carried out. In particular, the method is then carried out after switching the polarity of the magnetic field that is always required for the magnetic-inductive flow measurement, in particular in the time domain of a non-stationary magnetic field, as these time domains are not very suitable for a flow measurement

The standard deviation can be calculated as a statistical scattering measure. The calculation of the standard deviation is simple, and it has been found that in many cases the standard deviation shows a good correlation with the volume fraction of the second phase of the medium.

The first electrical parameter of the medium can be the electrical conductivity of the medium and/or the absolute value of the impedance of the medium and/or the phase angle of the impedance of the medium, in particular wherein the electrical conductivity is determined from the real part of the impedance of the medium, taking into account the geometry of the measurement volume in which the medium is located.

A significant, even more extensive insensitivity to a change in the material properties of the phases involved and their values for the first electrical parameter of the phases can be implemented by normalizing the determined scattering value of the first electrical parameter to the absolute value of the first electrical parameter, in particular to an absolute mean value of the first electrical parameter calculated from the plurality of determined values for the first electrical parameter of the medium. Standardization also makes the standard deviation independent of the absolute values and possible fluctuations in the absolute values of the first electrical parameter of the phases. Even if the material composition of one or both of the phases involved changes, there is still a good correlation between the change in the determined scattering value of the first electrical parameter and the volume fraction of the second phase of the medium when using standardized values.

A plurality of values for a second electrical parameter of the medium can be determined from the plurality of excitation signals and the plurality of reaction signals, in that a scattering value of a statistical scattering measure is determined from the plurality of determined values for the second electrical parameter of the medium, and that the volume fraction of the second phase in the medium can be determined using a mathematical relationship between the statistical scattering measure of the second electrical parameter and the volume fraction of the second phase in the medium with the determined scattering value of the second electrical parameter. The relationship to the volume fraction of the second phase in the medium (and thus also to the volume fraction of the first phase in the medium) to the value of the statistical scattering measure used of various electrical parameters of the medium has proven to be widely applicable with regard to various electrical parameters, so that other electrical parameters can also be readily used to determine the volume fraction of the second phase of the medium. This results in a wide range of possibilities for the mutual verification of calculation results, i.e. also with regard to the redundancy and/or combination of different calculation results in different ways and also possibilities with regard to the mutual verifiability and plausibility checks of results obtained in different ways with regard to the volume fraction of the second phase of the medium.

The aforementioned exemplary design of the method offers the possibility with regard to a further development of the method that an averaged volume fraction of the second phase of the medium can be calculated from the volume fraction of the second phase of the medium determined with the first electrical parameter of the medium and from the volume fraction of the second phase of the medium determined with the second electrical parameter of the medium. The possibility of combining volume fractions of the second phase of the medium determined on the basis of different electrical parameters of the medium lends itself because the relationship between the value of the used statistical scattering measure of an electrical parameter of the medium and the volume fraction of the second phase of the medium has proven to be relatively universal.

The volume fraction of the second phase in the medium determined with the first electrical parameter of the medium and/or the volume fraction of the second phase of the medium determined with the second electrical parameter of the medium and/or the averaged volume fraction of the second phase in the medium can be signaled, in particular, the determined volume fraction can be stored in a memory of a control and evaluation unit of the measuring device and/or displayed and/or transmitted to a connected communication partner via a communication interface of the measuring device of a flowmeter, in particular a magnetic-inductive flowmeter.

When a first limit value is exceeded by the volume fraction of the second phase in the medium determined with the first electrical parameter of the medium and/or when a second limit value is exceeded by the volume fraction of the second phase of the medium determined with the second electrical parameter of the medium and/or when a third limit value is exceeded by the averaged volume fraction of the second phase in the medium, the presence of a two-phase flow may be signaled, for example, can be stored in a memory of the control and evaluation unit of the measuring device and/or can be displayed and/or can be transmitted to a connected communication partner via a communication interface of the measuring device, in particular of a magnetic-inductive flowmeter.

The mathematical relationship between the statistical scattering measure of the first electrical parameter and the volume fraction of the second phase of the medium and/or the mathematical relationship between the statistical scattering measure of the second electrical parameter and the volume fraction of the second phase of the medium can be determined, wherein the scattering value of the first electrical parameter and/or the scattering value of the second electrical parameter can be determined for at least two different but known volume fractions of the second phase, in particular wherein a linear dependence is assumed between the statistical scattering measure of the first electrical parameter and the volume fraction of the second phase of the medium and/or between the statistical scattering measure of the second electrical parameter and the volume fraction of the second phase of the medium. If there are only two supporting points, then a linear equation can be derived immediately as the relationship between the volume fraction of the second phase of the medium and the scattering value of the statistical scattering measure of the second medium. If there are more than two supporting points, the relationship can be formulated linearly as a regression line. It has been shown that the use of such linear relationships represents a suitable approximation of the actual behavior.

More than two sampling points can be recorded, i.e. more than two correlations between the statistical scattering measure of an electrical parameter and the volume fraction of the second phase of the medium, and a polynomial description of the correlation can be determined as the mathematical correlation between the statistical scattering measure of an electrical parameter and the volume fraction of the second phase of the medium, or a description of the correlation is determined using spline interpolations, for example, by optimizing the descriptions of the correlations using the minimization of a deviation measure.

The object derived is also achieved by designing the control and evaluation unit in such a way that the measuring device carries out the method described in detail above during operation. In particular, the method is carried out with a flowmeter, especially with a magnetic-inductive flowmeter, in which the required electrodes are already provided anyway, or with a Coriolis mass flowmeter, a vortex flowmeter, a magnetic resonance flowmeter, or a variable area flowmeter. In the latter flowmeters, the electrodes required for carrying out the method are not necessarily implemented; they may have to be provided additionally.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows schematically, partial aspects of the principles of a method for determining the volume fraction of a phase of a multiphase medium,

FIG. 2 shows schematically, the steps of a method for determining the volume fraction of a phase of a multiphase medium,

FIG. 3 shows schematically, the method for determining the volume fraction of a phase of a multiphase medium on the basis of electrical conductivity,

FIG. 4 shows schematically, the method for determining the volume fraction of a phase of a multiphase medium on the basis of electrical impedance,

FIG. 5 shows schematically, the method for determining the volume fraction of a phase of a multiphase medium using the absolute value of the electrical impedance,

FIG. 6 shows schematically, the method for determining the volume fraction of a phase of a multiphase medium using the phase information of the electrical impedance, and

FIG. 7 shows schematically, a measuring device for determining the volume fraction of a phase of a multiphase medium using the methods described above.

### DETAILED DESCRIPTION

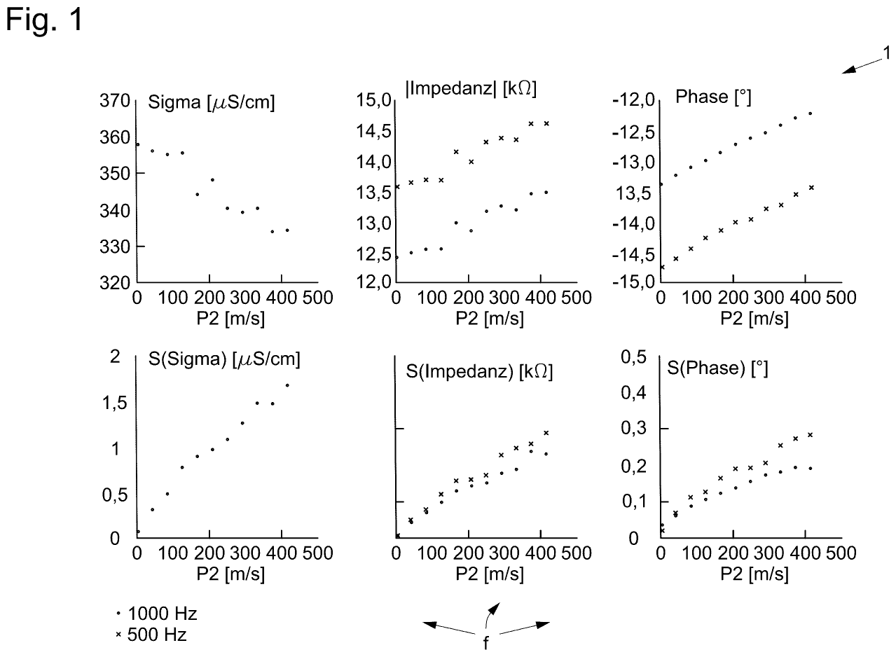

FIGS. 1 to 7 show in different aspects a method 1 and a measuring device 2 for determining the volume fraction V % of a phase of a multiphase medium M, wherein the medium M has a first phase P1 with a first value E1 for a first electrical parameter E and at least temporarily a second phase P2 with a second value E2 for the first electrical parameter E.

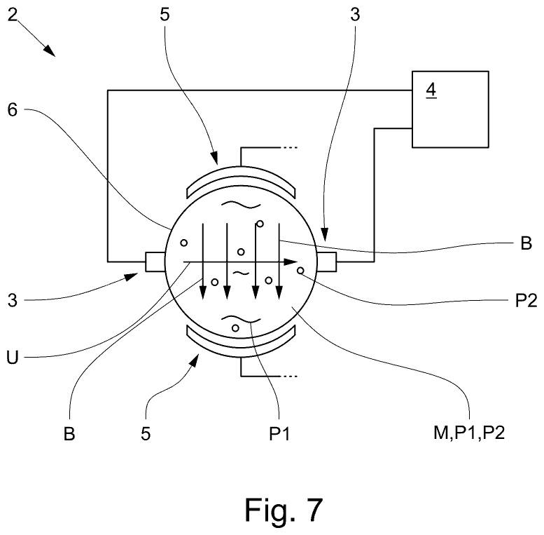

The measurements under consideration are frequently encountered measurement tasks in which the proportion of the various phases P1, P2 in the medium M is of interest. If, for example, a volume flow is measured at the same time, the information about the phase proportions of the phases P1, P2 in the medium M can be decisive for the measuring process, for example for the purpose of billing for delivered raw materials; this is immediately obvious if, for example, the first phase P1 of the medium M is liquid crude oil and the second phase P2 of the medium M is water (FIG. 7).

While FIGS. 1 to 6 illustrate more procedural features, FIG. 7 shows a measuring device 2 for determining the volume fraction V % of a phase of the multiphase medium M.

To determine the volume fraction V % of a phase of the multiphase medium M, it is known to impinge a plurality of electrical excitation signals on the medium M via a pair of electrodes 3 and to capture a plurality of corresponding electrical response signals for the electrical excitation signals. A plurality of values En for the first electrical parameter E of the medium M is determined from the plurality of excitation signals and the plurality of reaction signals. If it is a matter of known phase components P1, P2 with known values for the first electrical parameter E, for example the specific electrical conductivities, for the phases P1, P2 in pure form, then it is obvious that a changing proportion of the phases P1, P2 in the medium M also leads to a changing value for the first electrical parameter E of the medium M, and a determination of the first electrical parameter E of the medium M also enables a determination of, for example, the volume fraction V %_P2 of the second phase P2 of the medium M.

The measuring device 2 in FIG. 7 is a magnetic-inductive flowmeter with a measurement volume 6 used to hold a medium M, which in the present case is a measuring tube through which the medium M flows. The medium M has a first phase P1 with a first value E1 for a first electrical parameter E and, at least temporarily, a second phase P2 with a second value E2 for the first electrical parameter E. The measuring device 2 has a control and evaluation unit 4 and a pair of electrodes 3 in contact with the medium M, wherein the control and evaluation unit 4 generates a magnetic field B in the operating state of the measuring device 2 to fulfill the primary measuring task, namely the measurement of a volume flow rate through the measuring tube 6, by means of a magnetic field generator 5, by means of which—assuming a conductive medium M—a measuring voltage U is generated in the medium M which is dependent on the flow velocity and proportional to the flow velocity. This measuring voltage U is tapped via the electrode pair 3. In this way, flow information is obtained using the measuring device 2, which in the example is a magnetic-inductive flowmeter. The magnetic-inductive flowmeter is a typical example of a measuring device 2 that already has a pair of electrodes 3, although this pair of electrodes 3 has a different function in the original measuring task—the flow measurement—namely the passive recording of the induced measuring voltage U in the medium M.

Measuring devices 2 do not have to perform an additional measuring task as in the example shown in FIG. 7; they can also simply serve to determine the volume fraction V % of a phase of the multiphase medium M.

To determine the volume fraction V % of a phase of the multiphase medium M, the medium M is impinged with a plurality of electrical excitation signals via the pair of electrodes 3 and a plurality of corresponding electrical reaction signals are captured in addition to the electrical excitation signals. A plurality of values En for the first electrical parameter E of the medium M is determined from the plurality of excitation signals and the plurality of reaction signals. In the prior art, for example, the conductivity of the medium M is determined as the first electrical parameter E and the volume fraction V % of the involved phases P1, P2 of the medium M is inferred from the determined conductivity of the medium.

The direct determination of the volume fraction V % of a phase of the medium M has various disadvantages, some of which are associated with a material change in at least one of the participating phases of the medium M in the measurement volume 6 under consideration and thus with a change in the properties of the first electrical parameter E of the participating phases P1, P2.

The method 1 described here takes a significantly different approach. The method 1 provides for a scattering value S(En) of a statistical scattering measure S to be determined from the plurality of determined values En for the first electrical parameter E of the medium M, and for the volume fraction V %_P2 of the second phase in the medium M to be determined using a mathematical relationship f between the statistical scattering measure S of the first electrical parameter E and a volume fraction V %_P2 of the second phase in the medium M with the determined scattering value S(En) of the first electrical parameter.

The insight underlying method 1 is that not only the direct value—as known from the prior art—of the first electrical parameter E of the medium M provides information about the volume fraction V %_P2 of the second phase P2 in the medium, but surprisingly also the scattering value S(En) of the statistical scattering measure S, based on a plurality of measured values of the first electrical parameter E. This makes the determination of the volume fraction V %_P2 of the second phase P2 in the medium M to a certain extent independent of the absolute values for the first electrical parameter E of the medium M.

The relationships on which the method 1 is based are illustrated in FIG. 1 using real measurements with a magnetic-inductive flowmeter 2, as shown in FIG. 7. Various measurements have been performed for the first electrical parameter E of the medium M. On the one hand, the conductivity sigma of the medium M has been determined as the first electrical parameter E and, on the other hand, the impedance of the medium, which is an alternating current variable and as such has an absolute value (abs (impedance)) and a phase. These measurements are shown in the diagrams in the top line of FIG. 1. In the bottom line of FIG. 1, the standard deviations S of a plurality of measurements En of the corresponding first electrical parameter E in the form of the electrical conductivity sigma and the absolute value and phase of the impedance of the medium M are shown. The measurements have been performed at different frequencies.

The measurements in FIG. 1 have been performed in such a way that the admixture of the second phase P2 of air (gaseous) has always been increased at a constant volume flow of the first conductive phase P1 of the medium (water, liquid at 2.4 l/s). In this respect, the change in the volume flow of a phase also corresponds to a change in the volume proportion of this phase. The diagram shows measurements of the conductivity sigma of the medium M as well as the complex-value impedance of the medium, divided here into the absolute value of the impedance and the phase of the impedance. For each admixture of the second phase (air) P2 of the medium M, a plurality of values En of the electrical parameters E of the medium M have been captured. From the plurality of determined values En for the first electrical parameter E of the medium M, a scattering value S(En) of the statistical scattering measure S is then determined. In the present case, the standard deviation was selected as the statistical scattering measure S. That there is a relationship f—in many cases often linearly able to be approximated—between the volume fraction V %_P2 of the second phase in the medium M (which arises directly from the admixed proportion of the air of the second phase P2) and the standard deviation S of the corresponding values S(En) for the first electrical parameter E is surprising but nevertheless recognizable.

In the methods 1 carried out here, the excitation signal is an electrical voltage or an electrical current and the electrical response signal is consequently an electrical current or an electrical voltage. If the electrical parameters E of the medium M are alternating current quantities, such as the impedance or the admittance, then the excitation signal is usually a harmonic oscillation with an associated excitation frequency.

The impingement of the medium M with the electrical excitation signals takes place in a time domain in which no other measured value acquisition is performed in the medium M, wherein specifically in the magnetic-inductive flowmeter 2 according to FIG. 7, the measurements are performed after switching the polarity of the magnetic field B, which is generated by the magnetic field device 5, namely in the time domain of a non-stationary magnetic field B. If a measuring device is used to carry out method 1 which has no other measuring task at all, i.e. which is only used to determine the volume fraction V % of a phase of a multiphase medium M, or which only has a measuring task which does not physically collide with the determination of the volume fraction, then it is of course not necessary to pay attention to a possible collision with another measured value acquisition.

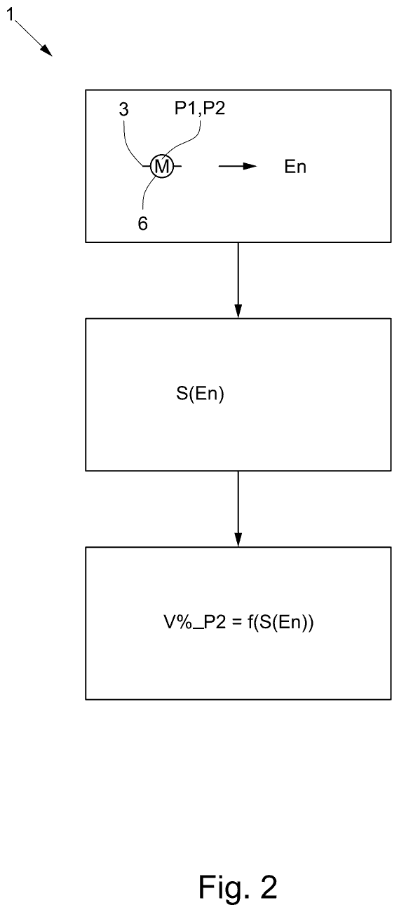

FIG. 2 shows the essential method steps for carrying out method 1. In the top-most method step, it is shown that the multiphase medium M with the phases P1 and P2 is located in a measurement volume 6. A pair of electrodes 3 is used to apply a plurality of electrical excitation signals to the medium M and a plurality of corresponding electrical response signals are captured in addition to the electrical excitation signals. The signals are used to determine a plurality of values En for the first electrical parameter of each medium, for example the electrical conductivity, the impedance, the admittance, etc. In the middle method step, a scattering value S(En) of the statistical scattering measure S, for example the standard deviation, is determined from the plurality of values En of the first electrical parameter E. Finally, in the last method step, the specific volume fraction V %_P2(S(En)) of the second phase P2 is determined using the mathematical relationship f between the statistical scattering measure S or the scattering value S(En) determined from it and the volume fraction V %_P2 of the second phase in the medium M.

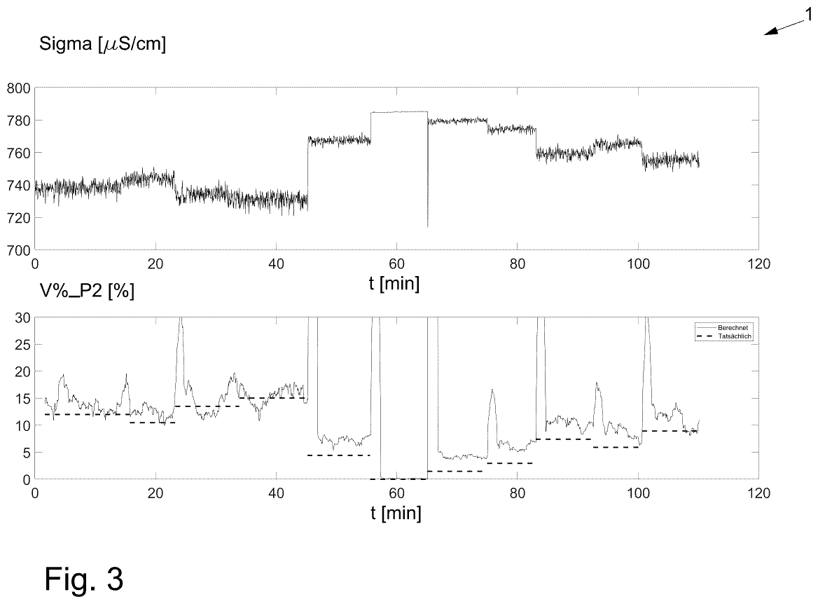

FIGS. 3 to 6 show measurement results obtained with method 1 with regard to the volume fraction V %_P2 of the second phase P2 of the medium M. The upper diagram of each figure shows the measured values of the first electrical variable over the time t over a relatively large period of almost two hours. The lower diagram shows the corresponding course of the actual (dashed line) and the determined volume fraction V %_P2 of the second phase P2 of the medium M. The actual course of the volume fraction V %_P2 of the medium M is constant over large time domains. The measurements were made in a measuring arrangement that allows the volume fractions V % of the phases P1, P2 of the medium M to be specified precisely. The measuring device 2 used is a magnetic-inductive measuring device as shown in FIG. 7, whose control and evaluation unit 4 is designed in such a way that it can carry out the method 1 described above during operation.

In the method 1 according to FIG. 3, the electrical conductivity sigma of the medium M is used as the first electrical parameter E of the medium M.

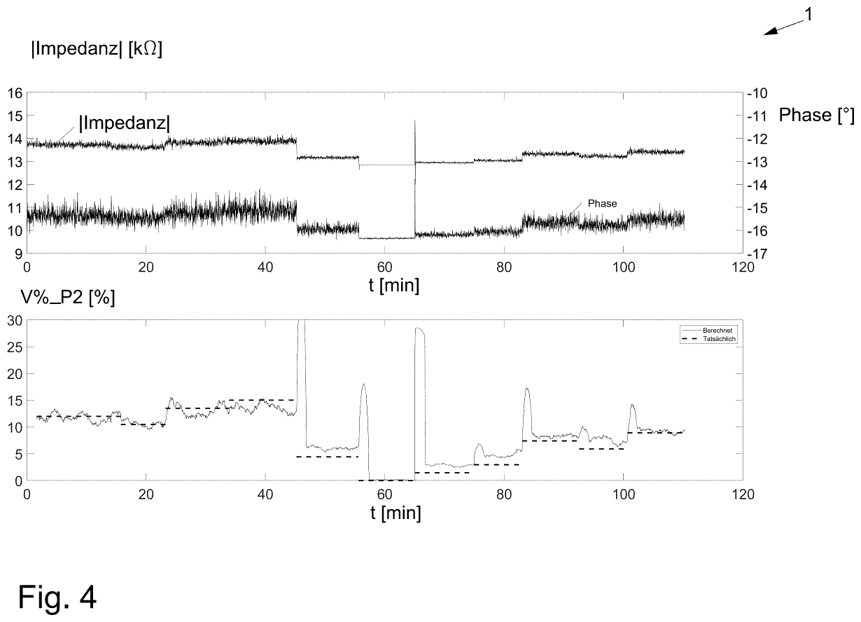

In the method 1 according to FIG. 4, the first electrical parameter E of the medium M is the impedance of the medium M, i.e. a bivalent electrical parameter which may be defined in connection with harmonic alternating quantities as excitation and reaction signals. In this respect, the absolute value of the impedance and the phase of the impedance are shown in the upper representation of FIG. 4.

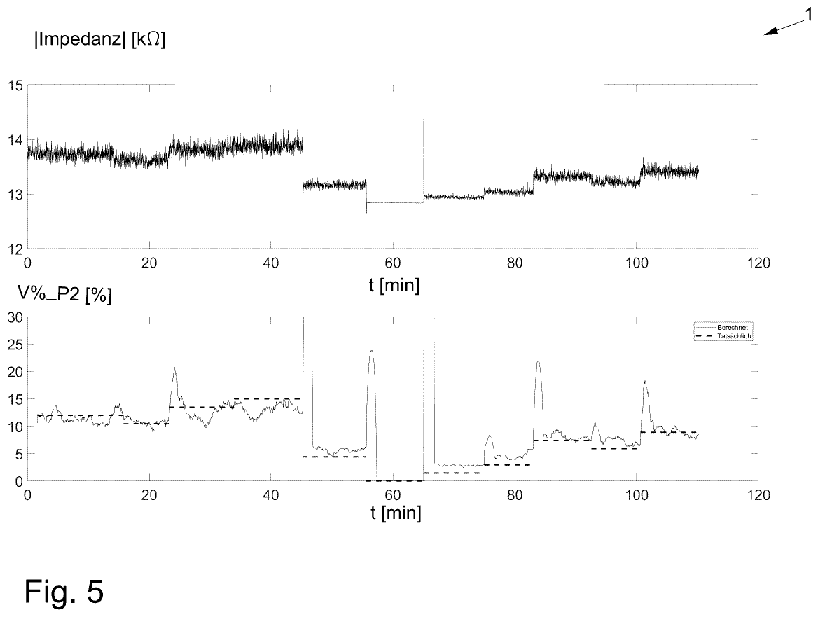

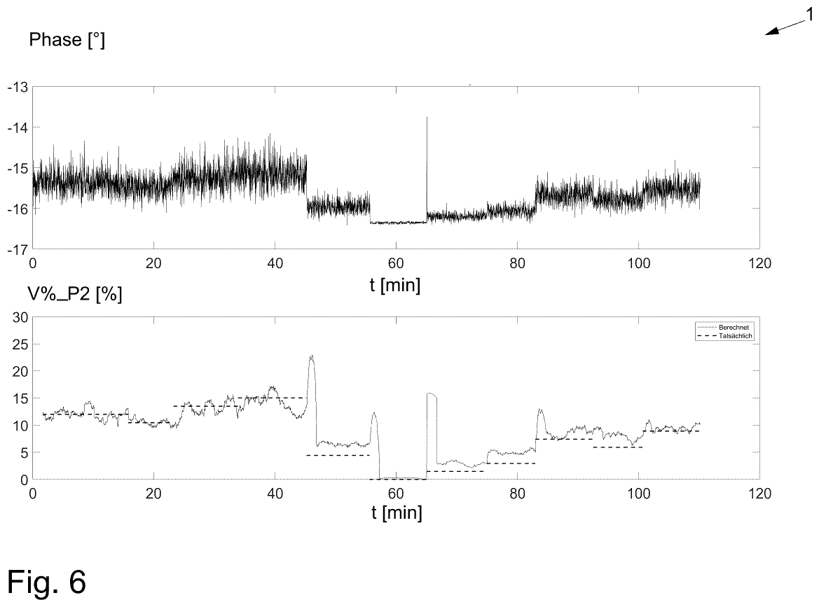

In FIGS. 5 and 6, only one of the two quantities of the complex-valued impedance is used, in FIG. 5 the absolute value of the impedance and in FIG. 6 the phase of the impedance.

In any case, it can be seen from the illustrations that the method based on the use of a value S(En) of a statistical scattering measure S based on determined values En for the first electrical parameter E is very well suited to determine the volume fraction V %_P2 of the second phase P2 of the multiphase medium M also quantitatively.

In the methods 1 according to FIGS. 3 and 5, the determined scattering value S(En) of the first electrical parameter E (electrical conductivity, absolute value of the impedance) has also been standardized to the absolute value of the first electrical parameter E, in the present case to an absolute mean value of the first electrical parameter E calculated from the plurality of determined values En for the first electrical parameter E of the medium M. This achieves a considerable insensitivity to material changes in the phases P1, P2 of the medium M, as the standardization may mean that the absolute values of the first electrical parameter are no longer important. Such standardization of the phase angle is of course not necessary, as this can only ever lie within a limited absolute range, irrespective of the material properties of the medium.

In an exemplary implementation of the method 1, a plurality of values for a second electrical parameter of the medium M is determined from the plurality of excitation signals and the plurality of reaction signals. If, for example, the magnitude of the impedance is used as the first electrical parameter, the phase angle of the impedance could be used as the second electrical parameter. A scattering value of a statistical scattering measure is then also determined from the plurality of values determined for the second electrical parameter of the medium M. Using a mathematical relationship between the statistical scattering measure of the second electrical parameter and the volume fraction of the second phase in the medium M, the determined scattering value of the second electrical parameter is used to determine the volume fraction of the second phase in the medium. In this example, it makes sense to calculate an averaged volume fraction of the second phase of the medium from the volume fraction V %_P2 of the second phase P2 of the medium M determined with the first electrical parameter E of the medium M and from the volume fraction of the second phase of the medium determined with the second electrical parameter of the medium.

The method 1 implemented in the measuring device 2 provides that the volume fraction V %_P2 of the second phase P2 in the medium M determined with the first electrical parameter E of the medium M and/or the volume fraction of the second phase of the medium M determined with the second electrical parameter of the medium and/or the averaged volume fraction of the second phase P2 in the medium M is signaled, namely is optionally stored in a memory of the control and evaluation unit 4 and/or is displayed and/or is transmitted to a connected communication partner via a communication interface of the measuring device 2.

The implementation of the method 1 in the measuring device 2 also provides for the presence of a two-phase flow to be signaled when a first limit value is exceeded by the volume fraction V %_P2 of the second phase P2 in the medium M determined using the first electrical parameter E of the medium M and/or when a second limit value is exceeded by the volume fraction of the second phase of the medium M determined using the second electrical parameter of the medium and/or when a third limit value is exceeded by the averaged volume fraction of the second phase in the medium, namely is optionally stored in a memory of the control and evaluation unit 4 and/or is displayed and/or is transmitted to a connected communication partner via a communication interface of the measuring device 2.

Taking into account the illustration of the functional relationships f in FIG. 1 (lower three figures), it is understandable that the mathematical relationship f between the statistical scattering measure S of the first electrical parameter E and the volume fraction V %_P2 of the second phase of the medium M has been determined wherein the scattering value S(En) of the first electrical parameter E has been determined for at least two different but known volume fractions V %_P2 of the second phase P2 of the medium M, wherein the volume fractions V % in FIG. 1 correspond to the flow components of the second phase P2 of the medium M. In the examples, a linear dependence between the statistical scattering measure S of the first electrical parameter E and the volume fraction V %_P2 of the second phase P2 of the medium M has been adopted.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for determining a volume fraction of a phase of a multiphase medium, the medium having a first phase with a first value for a first electrical parameter and, at least temporarily, a second phase with a second value for the first electrical parameter, the method comprising:
impinging the medium with at least two electrical excitation signals via a pair of electrodes;
capturing at least two corresponding electrical reaction signals for the electrical excitation signals;
determining at least two values for the first electrical parameter of the medium from the at least two excitation signals and the at least two reaction signals;
determining a scattering value of a statistical scattering measure from the at least two determined values for the first electrical parameter of the medium;
determining the volume fraction of the second phase in the medium using a mathematical relationship between the statistical scattering measure of the first electrical parameter and a volume fraction of the second phase in the medium with the determined scattering value of the first electrical parameter.

2. The method according to claim 1, wherein the excitation signal is an electrical voltage or an electrical current, and the electrical response signal is an electrical current or an electrical voltage, or wherein the excitation signal is a harmonic oscillation with an excitation frequency.

3. The method according to claim 1, wherein the impingement of the medium with the electrical excitation signals takes place in a time domain in which no other measured value acquisition is carried out in the medium, wherein, in a case of a magnetic-inductive measuring device, no magnetic-inductive flow measurement is carried out, then takes place after a switching of the polarity of the magnetic field or in the time domain of a non-stationary magnetic field.

4. The method according to claim 1, wherein a standard deviation is calculated as the statistical scattering measure.

5. The method according to claim 1, wherein the first electrical parameter of the medium is the electrical conductivity of the medium and/or an absolute value of the impedance of the medium and/or a phase angle of the impedance of the medium, or wherein the electrical conductivity is determined from a real part of the impedance of the medium, taking into account a diameter of a measurement volume for the medium.

6. The method according to claim 1, wherein the determined scattering value of the first electrical parameter is normalized to the absolute value of the first electrical parameter or to an absolute mean value of the first electrical parameter calculated from the at least two determined values for the first electrical parameter of the medium.

7. The method according to claim 1, wherein at least two values for a second electrical parameter of the medium is determined from the at least two excitation signals and the at least two reaction signals, wherein a scattering value of a statistical scattering measure is determined from the at least two determined values for the second electrical parameter of the medium, and wherein, using a mathematical relationship between the statistical scattering measure of the second electrical parameter and the volume fraction of the second phase in the medium, the volume fraction of the second phase in the medium is determined using the determined scattering value of the second electrical parameter.

8. The method according to claim 7, wherein an averaged volume fraction of the second phase of the medium is calculated from the volume fraction of the second phase of the medium determined with the first electrical parameter of the medium and from the volume fraction of the second phase of the medium determined with the second electrical parameter of the medium.

9. The method according to claim 1, wherein the volume fraction of the second phase in the medium determined with the first electrical parameter of the medium and/or the volume fraction of the second phase of the medium determined with the second electrical parameter of the medium and/or the averaged volume fraction of the second phase in the medium is signaled or is stored in a memory of a control and evaluation unit and/or is displayed and/or is transmitted to a connected communication partner via a communication interface of a measuring device.

10. The method according to claim 1, wherein when a first limit value is exceeded, the presence of a two-phase flow is signaled by the volume fraction of the second phase in the medium determined using the first electrical parameter of the medium and/or when a second limit value is exceeded, the volume fraction of the second phase of the medium determined using the second electrical parameter of the medium and/or when a third limit value is exceeded, the presence of a two-phase flow is signaled by the averaged volume fraction of the second phase in the medium or is stored in a memory of a control and evaluation unit and/or is displayed and/or is transmitted to a connected communication partner via a communication interface of a measuring device.

11. The method according to claim 1, wherein the mathematical relationship between the statistical scattering measure of the first electrical parameter and the volume fraction of the second phase of the medium and/or the mathematical relationship between the statistical scattering measure of the second electrical parameter and the volume fraction of the second phase of the medium is determined in that the scattering value of the first electrical parameter and/or the scattering value of the second electrical parameter are determined for at least two different known volume fractions of the second phase of the medium, or wherein a linear dependence is assumed between the statistical scattering measure of the first electrical parameter and the volume fraction of the second phase of the medium and/or between the statistical scattering measure of the second electrical parameter and the volume fraction of the second phase of the medium.

12. The method according to claim 11, wherein more than two supporting points are recorded or more than two correlations between the statistical scattering measure of the first electrical parameter and the volume fraction of the second phase of the medium, and a polynomial description of the relationship is determined as the mathematical relationship between the statistical scattering measure of the electrical parameter and the volume fraction of the second phase of the medium or a description of the relationship is determined using spline interpolations by optimizing the descriptions of the relationships using the minimization of a deviation measure.

13. A measuring device comprising:
a measurement volume provided to accommodate a medium, the medium having a first phase with a first value for a first electrical parameter and, at least temporarily, a second phase with a second value for the first electrical parameter;
a control and evaluation unit; and
a pair of electrodes in contact with the medium,
wherein the control and evaluation unit, in an operating state of the measuring device, impinges at least two electrical excitation signals on the medium via the pair of electrodes and captures at least two corresponding electrical response signals for the electrical excitation signals,
wherein at least two values for the first electrical parameter of the medium is determined from the at least two excitation signals and the at least two response signals, and
wherein the control and evaluation unit is designed such that the measuring device carries out the method according to claim 1 during the operating state.

14. The measuring device according to claim 13, wherein the measuring device is a magnetic-inductive flowmeter with a pair of electrodes for recording an electrical voltage induced in the medium, and wherein the pair of electrodes is also used to impinge at least two electrical excitation signals on the medium.
